(19) United States
(12) Patent Application Publication
(10) Pub. No.: US 2008/0138833 A1
(43) Pub. Date: Jun. 12, 2008

(54) METHODS AND COMPOSITIONS FOR ASSESSING IL-12 OR THE NEUTRALIZATION OF IL-12 IN A SAMPLE

(75) Inventors: Ralph Patrick Braun, Highland Mills, NY (US); Giuseppe Palladino, Chester, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: WYETH, Madison, NJ (US)

(21) Appl. No.: 11/949,958

(22) Filed: Dec. 4, 2007

Related U.S. Application Data
(60) Provisional application No. 60/868,997, filed on Dec. 7, 2006.

(51) Int. Cl. G01N 33/53

(52) U.S. Cl. 435/7.1

(57) ABSTRACT

Methods for detecting the presence or amount of Interleukin-12 (IL-12), particularly human IL-12, or antibodies that neutralize the activity of human IL-12 in a human biological sample, involve incubating the biological sample with an NK cell line or progeny thereof. A correlation is determined between the presence or amount of IL-12 (or anti-IL-12 antibodies) in the sample and the amount of gamma interferon (or a precursor activation factor or cytokine) secreted by the NK cells. For measuring antibodies, the sample is pre-incubated with IL-12. Levels of a cytokine or activation factor, such as IFN-γ, secreted by the NK cells when in the presence of IL-12 are then compared with a control level of the cytokine or activation factor secreted by the NK cells or progeny thereof in the presence of a known amount of IL-12 and without the biological sample. The presence of IL-12 or neutralizing anti-IL12 antibodies in the sample is indicated by a variation, e.g., an elevation or reduction, in the level of the secreted cytokine or activation factor in the sample as compared to the control level. These methods are useful for monitoring or determining the presence of IL-12 in a sample or neutralizing antibodies generated to IL-12 when the IL-12 is delivered as a vaccine adjuvant and in clinical situations wherein IL-12 enhancement or diminution is involved, among others.

### CROSS-REFERENCE TO RELATED APPLICATIONS

This application claims the benefit of the priority of U.S. Provisional Patent Application No. 60/868,997, filed Dec. 7, 2006.

### BACKGROUND OF THE INVENTION

A common problem with biologicals, e.g., proteins and proteinaceous materials, used as medicines is the generation of immune responses in the host against the molecule of interest (Korean, E. et al, 2002 Curr. Pharm. Biotechnol., 3:349-60; Mire-Sluis, A. R., et al, 2004, J. Immunol. Meth., 289:1-16). Unwanted immune stimulation has been observed in hosts in response to several protein therapeutics, such as interferons and GM-CSF (Wadhwa, M. et al, 2003, J. Immunol. Meth., 278:1-17). An unwanted host response to an administered biological may reduce the potency of the delivered biological and disrupt the activity of the host's endogenous protein.

An exemplary biological for which adverse immune reactions must be assessed is Interleukin (IL-12). IL-12 is an important cytokine in the development of cellular and innate immune responses, having as its key activity the promotion of the Th1 helper response and the enhancement of IFN-γ secretion. This ability of IL-12 to influence cellular immune responses makes it of great interest for medical interventions, e.g., against cancer and myasthenia gravis, and specifically for inclusion as an adjuvant in a variety of vaccine formulations (Gately, M. K. et al, 1995 Measurement of Human and Mouse Interleukin-12, p. 6.16.1-6.16.15. In A. M. K. John E. Coligan, David H. Margulies, Ethan M. Shevach, Warren Strober (ed.), Current Protocols in Immunology. John Wiley & Sons, Inc.; Boyer, J. D. et al, 2005 J. Med. Primatol., 34:262-70; Chattergoon, M. A. et al, 2004, Vaccine 22:1744-50; Cooper D. et al, 2004 Vaccine, 23:236-46; Egan, M. A. et al, 2005 AIDS Res. Hum Retroviruses, 21:629-43). IL-12, when employed in vaccine formulations as an adjuvant, improves the efficacy of the vaccine, especially for a vaccine that depends on a cellular immune response. Although the amount of IL-12 delivered as a vaccine adjuvant is low, it is within a range intended to generate an immune response. Thus, even when used as an adjuvant, IL-12 in any vaccine formulation is recognized by the immune system. Therefore, it is important to identify and/or quantify IL-12 or an antibody response to IL-12 in a vaccinated subject, especially upon repeated use of the vaccine.

Two types of assays are generally used to evaluate the presence of antibodies. A direct binding assay measures the physical presence or amount of antibodies recognizing a designated antigen in a sample, e.g., an enzyme-linked immunoassay (ELISA). In one exemplary simple format, an ELISA for the measurement of anti-IL-12 antibodies employs an excess amount of immobilized IL-12 that binds anti-IL-12 target antibody in a sample exposed to the immobilized IL-12. Subsequently, a detector antibody that binds to the immobilized antibody is introduced. A detectable signal is generated by the interaction of the detector antibody with a selected signal-generating label that permits the measurement of the amount of anti-IL-12 antibody present in the sample. Such direct binding assays tend to be simple, reproducible and capable of screening large numbers of samples. However, direct binding assays, particularly cytokine ELISAs, have frequently been susceptible to nonspecific binding and often lead to false positives. More importantly, direct binding assays do not measure potential biological activity of the antibodies.

The second assay type is a neutralization assay which measures the presence and/or amount of both binding and functional antibodies in a sample based upon neutralization of a biological effect of the IL-12 (Mire-Sluis, A. R. 2001, Pharm. Res., 18:1239-46; Mire-Sluis et al, 2004, cited above; Wadhwa et al, 2003, cited above). For example, the current method of choice for quantifying neutralizing human IL-12 antibody involves measuring the proliferation of human peripheral blood mononuclear cell (PBMC) blasts in the presence of IL-12 (Gately, M. K, et al, 1995 Measurement of Human and Mouse Interleukin-12, p. 6.16.1-6.16.15. In A. M. K. John E. Coligan, David H. Margulies, Ethan M. Shevach, Warren Strober (ed.), Current Protocols in Immunology. John Wiley & Sons, Inc.; see, also, R&D Systems, catalog AB-219-NA “Anti-human IL-12 Antibody”, published February 2004). Such biological assays can yield information on the biological activity of the antibodies. However, such biological assays are commonly highly variable, difficult and require use of human blood. In this instance, the variability is most likely due to the heterogeneity of the responding cell population. Similarly, when the assay is applied to a test method used to evaluate large number of samples, this variability would be a problem. Generally, such assays can test only a limited number of samples.

Another confounding circumstance arises because other cytokines also cause proliferation of human PBMC, and when a sample contains a mixture of cytokines, the results can be inaccurate. Further, current assays and methods to detect IL-12 neutralizing antibodies are used to characterize antibodies raised in non-human species, and such assays have not been optimized for screening human serum samples.

Thus, there is a need in the art for methods and compositions that permit a rapid, complete and accurate assessment (both quantitative and qualitative) of human IL-12-specific neutralizing antibodies or IL-12 itself in human serum.

### SUMMARY OF THE INVENTION

Methods for detecting the presence or quantity of Interleukin-12, or antibodies that neutralize the activity of human IL-12, in a human biological sample are described herein.

In one embodiment, a method for measuring the presence or amount of human IL-12 in a biological sample involves incubating a Natural Killer (NK) cell line or progeny thereof with the sample. Levels of a desired activation factor or cytokine, such as IFN-γ secreted by the cells in response to the presence of IL-12 are measured. The levels of the desired factor, e.g., IFN-γ, secreted by said cells in said sample are then compared with a reference standard to detect the presence or amount of IL-12 in the sample. The reference standard is typically a dosage curve of the factor secreted by the same cells in the presence of one or more known amounts of IL-12.

In another embodiment, a method for measuring the presence or amount of neutralizing antibodies to human IL-12 in a biological sample involves incubating a natural killer (NK) cell line or progeny thereof, with the biological sample that has been pre-incubated with IL-12. As disclosed herein, one exemplary NK cell line is the NK-92MI cell line (ATCC Accession No. CRL-2408). Levels of a desired activation factor, such as IFN-γ, secreted by the cells are then obtained and compared with a control level of that activation factor secreted by cells of the NK cell line or progeny thereof incubated in the presence of a known amount of IL-12. The presence of neutralizing anti-IL12 antibodies in the sample is indicated by a reduction in the level of secreted activation factor, e.g., IFN-γ, in the sample as compared to that activation factor secreted by control cells. In one embodiment, conditions used in this method permit the cells to produce IFN-γ in response to IL-12. In one embodiment, the pre-incubation occurs under conditions sufficient to allow IL-12 to complex with any neutralizing IL-12 antibody in the samples.

In another embodiment, a neutralization assay method for detecting antibodies that neutralize the activity of human IL-12 in a human biological sample includes the step of incubating NK cells or progeny thereof with a sample from a human subject both prior to, and after, administration of a composition comprising IL-12 to the subject. Each sample is pre-incubated with IL-12. Thereafter, the levels of IFN-γ secreted by cells in the pre-administration samples are compared with the levels of IFN-γ secreted by such cells in the post-administration samples. In this method, the presence of neutralizing anti-IL12 antibodies in the post-administration samples is indicated by a reduction in the level of secreted IFN-γ as compared to the pre-administration sample IFN-γ level.

In still another embodiment, a neutralization assay method for detecting antibodies that neutralize the activity of human Interleukin-12 in a human biological sample includes adding a known amount of IL-12 to dilutions of the sample (either single point dilutions or serial dilutions) and incubating the dilutions. To each dilution is added a known number of cells of an NK cell line or progeny thereof and the dilutions are incubated for a time sufficient to allow the cells to secrete IFN-γ. The levels of IFN-γ secreted by the cells in each dilution are compared with a control IFN-γ level secreted by the NK-92MI cells in the presence of a known amount of IL-12 and without the sample. The presence of neutralizing anti-IL12 antibodies in the dilutions is indicated by a reduction in the level of secreted IFN-γ in the dilutions as compared to the control IFN-γ level.

In a further embodiment, a high-throughput, automated method for detecting antibodies that neutralize the activity of human IL-12 in a large number (>100) of human biological samples involves diluting (either single-point dilutions or serial dilutions) a biological sample with culture medium in multiple wells on an assay plate. A known amount of IL-12 is added to each well and the wells are incubated for a time sufficient to allow the IL-12 to complex with any neutralizing IL-12 antibodies in the wells. To each well is added a known number of NK-92MI cells or progeny thereof. The wells are incubated for a time sufficient to allow the cells to secrete IFN-γ. Thereafter, the levels of IFN-γ secreted by the cells in each well are measured. The IFN-γ level in each well is compared with a control IFN-γ level secreted by the NK-92MI cells in the presence of a known amount of IL-12 and without the sample. The presence of neutralizing anti-IL12 antibodies in the wells is indicated by a reduction in the level of secreted IFN-γ in the wells as compared to the control IFN-γ level.

Other aspects and embodiment of the present invention are disclosed in the following detailed description.

### BRIEF DESCRIPTION OF THE DRAWINGS

FIG. 1 is a bar graph showing the response of NK-92MI cells to various cytokines. NK-92MI cells were cultured in the presence of 10 ng/mL of different cytokines, identified under the columns on the x axis. After overnight culture supernatants were tested for the presence of IFN-γ by an enzyme linked immunosorbent assay (ELISA), with results measured in optical density at 450 nm wavelength.

FIG. 2 is a graph showing the response of NK-92MI cells to different doses of IL-12 (♦) or IL-18 (▪) added to culture. IFN-γ levels were measured by an ELISA from supernatants of overnight cultures of NK-92MI cells (30,000 per well) incubated with varying doses (ng/mL) of IL-12 or IL-18.

FIG. 3 is a graph showing the results of the IL-12 neutralization assay using commercially prepared antibodies specific for IL12. Anti-IL-12 antibody preparations purchased from R&D Systems (Minneapolis, Mn.) (▪) and PeproTech (Rocky Hill, N.J.) (▴) were serially diluted in a 96-well plate prior to the addition of IL-12 to culture wells. Following a 1 hr incubation of antibody/cytokine, 30,000 NK-92MI cells were added to each well. The final IL-12 concentration after addition of the NK-92MI cells was 3 IU/mL (0.3 ng/mL). IFN-γ levels in overnight cell culture supernatants were measured by an ELISA in optical density at 450 nm wavelength.

FIG. 4 is a graph showing the results of an IL-18 neutralization assay. Negative serum pool (NSP; ♦) alone or with the addition of anti-IL-12 antibodies (▪) or anti-IL-18 antibodies (▴) were serially diluted in a 96-well plate prior to the addition of IL-18 to culture wells. Following a 1 hr incubation of antibody/cytokine, 60,000 NK-92MI cells were added to each well. The final IL-18 concentration after addition of the NK-92MI cells was 30 ng/mL. IFN-γ levels in overnight cell culture supernatants were measured by an ELISA in optical density at 450 nm wavelength.

FIG. 5 is a graph showing results of an IL-12 neutralization assay to test activity in various human sera. Human sera were serially diluted and tested in the IL-12 neutralization assay under standard conditions. IFN-γ secreted into overnight cultures was measured by an ELISA. The curve for commercial goat anti-IL-12 standard antibody (▴) is contrasted with four other curves representing four individual human sera (▪), and other symbols with the curves mostly overlayed). The assay was performed with 30,000 cells/well and final concentration of 3 IU/mL (0.3 ng/mL) of IL-12.

FIG. 6 is a graph showing the response of NK-92MI cells to different doses of Human (♦) and Rhesus (▪) IL-12 added to culture. IFN-γ levels were measured by an ELISA from supernatants of overnight cultures of NK-92MI cells (30,000 per well) incubated with varying doses (pg/mL) of IL-12.

FIG. 7 is a graph showing the results of the Rhesus IL-12 neutralization assay using commercially prepared antibodies specific for human IL-12 and Rhesus serum. Anti-IL-12 antibody preparations purchased from R&D Systems (Minneapolis, Mn.) were first diluted to 1:10 in either medium (♦), or Rhesus serum (▪), and then serially diluted in a 96-well plate prior to the addition of Rhesus IL-12 to culture wells. As well, a third test sample was Rhesus serum alone with no added IL-12 antibody (▴). Following a 1 hr incubation of antibody/cytokine, 30,000 NK-92MI cells were added to each well. The final Rhesus IL-12 concentration after addition of the NK-92MI cells was 0.03 ng/mL. IFN-γ levels in overnight cell culture supernatants were measured by an ELISA in optical density at 450 nm wavelength.

### DETAILED DESCRIPTION OF THE INVENTION

Novel IL-12 biological assays and neutralization assays are described that are useful to detect the presence of, or quantitatively measure, either human interleukin 12 (IL-12) present in certain biological samples, such as human serum, or antibodies that can neutralize the activity of human IL-12 in such samples. These assays employ a Natural Killer (NK) cell line with a consistent biological response to IL-12 and can be performed in a format to provide data within 24 hours.

The method for detecting IL-12 or antibodies that neutralize the activity of IL-12 in a biological sample requires a cell line or progeny thereof that exhibits a consistent biological response to the presence of IL-12. For example, following binding of IL-12 to its receptor on the NK cells, any cellular component that is altered in a dose dependent manner as a result of IL-12 binding may be used as an indicator of IL-12 levels. One example of this is the secretion of IFN-γ by cells of an NK cell line. Incubation of these cells with a sample that contains IL-12, or has been pre-incubated with IL-12, induces these cells to secrete the factor, e.g., IFN-γ. Thus, IL-12 in the sample may be evaluated by measuring the amount of IFN-γ secreted by the cells and comparing this amount to a reference standard. The reference standard is preferably predetermined or may be determined in situ. The reference standard is generally prepared by measuring the amount of IFN-γ secreted by the same cells under the same conditions using a series of known amounts of IL-12, e.g., a standard dosage curve. Thus, whether and how much IL-12 is in the experimental sample may be determined by comparing the secretion of IFN-γ in the experimental culture with the NK cell line with the reference standards. Typically the reference standard is a dosage curve, which facilitates the determination of the amount of IL-12 in the experimental sample.

Conversely, this method is also employed to identify the presence (or quantity) of any anti-IL-12 neutralizing antibody in a sample that binds the IL-12 and prevents or inhibits the inductive effect on the secretion of IFN-γ. When neutralizing antibody is being measured, the decrease in IFN-γ secretion in the presence of a known amount of IL-12 (caused by the binding of the IL-12 to the neutralizing antibody) enables the identification of the presence and quantity of anti-IL-12 antibodies in the sample. The levels of IFN-γ secreted by these cells when they are in the presence of a known amount of IL-12 and in a sample that has an unknown presence or quantity of anti-IL-12 neutralizing antibodies are thereafter compared with a reference standard, i.e., the level of IFN-γ secreted by the same NK cell line or progeny thereof in the presence of the same amount of IL-12 and an amount of reference standard anti-IL-12 antibody. The neutralizing anti-IL12 antibodies in the sample are indicated qualitatively by a reduction in the level of secreted IFN-γ in the sample and are quantified by comparison to the IFN-γ levels secreted by the cells in the presence of known amounts of anti-IL-12 reference standard antibody.

### A. The Cell Line

An important issue for neutralization assays is to choose an appropriate biological response that provides consistent dose-related results. Selection of the type of cell to produce the response in a reliable and measureable fashion is crucial. Mouse cells do not respond to human IL-12, which limits the number of potential cell types that can be used. Although a cell line responsive to human IL-12 (KIT225) has been described, it is considered to be more variable in its response than using human PBMC blasts (Gately, infra). The identification of a stable cell line was vital for the development of a reliable and sensitive bioassay.

The inventors determined that natural killer (NK) cell lines and their progeny, in general, demonstrate consistent, measurable biological responses to IL-12 by secreting or producing an activation factor or cytokine, such as IFN-γ, TCF-α or others, in measurable amounts. By “progeny” as used herein is meant cells derived or propagated from growth of a parent cell line and/or cells propagated by growth of a parent cell line which has been mutated to provide or enhance a biological, chemical or functional characteristic. Thus, a variety of NK cell lines are useful components in a neutralization assay to measure levels of IL-12.

In one embodiment, an NK cell line is NK-92 (ATCC Accession No. CRL-2407) which is an IL-2 dependent cell line. The NK-92 cell line is known to respond to human IL-12. If supplemented with IL-2 in its culture medium, NK-92 cells produce a consistent and readily measured response to IL-12 (Hodge, D. L. et al, 2002 J. Immunol. 168:6090-8; International Patent Publication No. WO 98/49268, published Nov. 5, 1998). Much of the work using the NK-92 cell line has been focused on its use as a cancer therapy (Grund and Muise-Helmericks 2005 J. Immunol. Meth., 296:31-6; Yao A. Y., et al, 2004 Cell Res. 14:155-60), or as a measure of cellular cytotoxicity assays (Costa C., et al, 2002 J. Immunol., 168:3808-16; Zhu, M., et al, 2005 Transplantation, 79:289-96). This parent cell line is expected to show similar IL-12 responsiveness to NK-92MI, as are other NK cell lines.

In another embodiment, an NK cell line, NK-92MI (ATCC Accession No. CRL-2408) was found to produce a consistent and readily measured response to IL-12. The NK-92MI cell line is an IL-2 independent progeny derived from the NK-92 cell line in which the gene for IL-2 has been inserted into the genome of the cell. The IL-2 independence was chosen to avoid the need to culture the cells in IL-2.

Other NK cell lines or subsets and their progeny that are anticipated to be useful in the assay described herein include those NK cells identified as CD56dimCD3− NK cells, CD56brightD3− NK cells, CD56−CD161+CD3− NK cells, CD56−[[CD16]+ NK cells, CD56+/CD83+/CCR7+/CD25+ cells, CD56+CD3−CD8− NK cells, CD56+CD3−CD8+ NK cells, CD56+CD3−CD25+ NK cells, CD56+CD3−CD25− NK cells, CD56+CD3−CD69+ NK cells, CD56+CD3−CD69− NK cells, CD56+CD3−CD94+ NK cells, CD56+CD3−CD94− NK cells, CD56+CD3−CD57+ NK cells, CD56+CD3−CD57− NK cells, CD56+CD3−CD16+ NK cells, CD56+CD3−CD16− NK cells. See, e.g., Mavilio et al, Proc. Natl. Acad. Sci. USA, (Feb. 7, 2005) Epub (PMID 15600323); Nguyen et al, J. Neuroimmunol. Aug. 10, 2006; Epub, PMID:16904757; Berahovich et al, 2006 J. Immunol, 144:7833-7840; Batoni et al, 2005 Scand. J. Immunol. 62(6):498-506; Bosch et al, 2005 Psychosom. Med. 67:366-375; Trotta et al 2005 Blood 105:3011-3018; Ritz J. 2005 Blood, 105:3003; Loxa and Perussia 2004 J. Immunol., 172:88-96; Vitale et al, 2004 Eur. J. Immunol. 34(6):1715-22; Cooper et al, 2001 Blood, 97:3146-3151; Bratke et al, 2005 Eur. J. Immunol. Epub. PMID 16106370; Addison et al, Immunol., 2005 116(3):354-61. These documents are incorporated by reference herein to provide additional information on other NK cell subsets useful in this assay.

For ease of discussion, the NK cell line is referred to specifically hereinafter as the exemplary NK-92MI cell line and progeny thereof to exemplify an embodiment of the neutralization assay described herein. However, this assay is not limited to the use of this one NK cell line, as discussed above. For use in the assays described herein the NK-92MI cells are grown in suitable media, such as that described in Example 1 below. However, one of skill in the art would be readily able to grow the cells in other known medium formulations.

Further, for ease of discussion, the cytokine or activation factor employed in the examples and description of the neutralization assay hereinafter is IFN-γ. However, this assay may be readily adapted to measure a different cytokine or activation factor produced or secreted by the NK cell lines in response to IL-12. For example, cytokines or factors that are intermediates in the biological and biochemical cascade that results in the production of IFN-γ are anticipated to produce the same effect as measurement of IFN-γ for use in the neutralization assay.

One of skill in the art by review of this specification will readily be able to substitute a variety of NK cell lines, as discussed herein, and a variety of suitable cytokines/factors, as discussed herein, to perform the neutralization assay. Thus, according to only one exemplary embodiment of the neutralization assay described in the examples below, anti-IL-12 neutralizing antibodies in a biological sample are detected by the reduction in IFN-γ secretion by NK-92MI cells and/or IL-12 in a biological sample are detected in reference to IFN-γ secretion by NK-92MI cells.

### B. The Biological Sample

The biological sample that may be examined using the biological/neutralization assay is any biological fluid or tissue that contains or is suspected of containing IL-12 or anti-IL-12 antibody. Such samples include fluids or tissues obtained from a mammalian subject, preferably a human subject. For example, such samples include, without limitation, a body fluid, such as whole blood, serum or plasma. Other samples may include urine, saliva, mucosal secretions, exudates, or tissue. Still other samples include laboratory test samples, such as cell cultures, or dilutions of the samples indicated above.

In one embodiment, the biological sample is obtained from a subject that has been administered a composition containing IL-12 protein, such as a pharmaceutical composition or immunogenic composition containing IL-12 protein. For example, the IL-12 protein may be present as the primary therapeutic, a secondary therapeutic or an adjuvant. See, e.g., D. Cooper et al, 2004 Vaccine, 23:236-246 which discusses the use of IL-12 protein to adjuvant an HSV vaccine; C. F. Eisenbeis et al, 2005 J. Clin. Oncol. 23(34):8835-44 which discusses the use of IL-12 protein and IFN-α2b in the treatment of advanced cancer; and C. B. O'Brien et al, 2001 Amer. J. Gastroenterol., 96:2473, which discusses the use of IL-12 in the treatment of chronic Hepatitis C infections, among others.

In another aspect, the sample is obtained from a subject that has been administered a composition expressing IL-12. Such compositions expressing IL-12 include, without limitation, DNA plasmids expressing IL-12. Still other such compositions expressing IL-12 include viral vectors, attenuated viral vectors (for example, adenovirus vectors and Vesicular Stomatitis Virus (VSV) vectors) and viral replicons. As a specific example, such biological samples may be serum or plasma obtained from a human subject who had received an administration of a viral replicon, such as a Venezuelan Equine Encephalitis Virus (VEE) replicon, that is engineered to express IL-12. Among such IL-12-expressing compositions are those described in M. A. Egan et al, 2005 AIDS Res. Hum. Retroviruses 21(7):629-43, which employed a plasmid-expressed IL-12 and a VSV-expressed IL-12.

In still another embodiment, a biological sample subject to the assay described herein is obtained from a human subject that has been administered a composition containing an IL-12-inducing agent. Among such IL-12 inducing agents are, without limitation, aspirin (M. Buckland et al, 2006 Int. Immunopharmacol, 6(11):1729-1735), alginic acid oligosaccharide (see, e.g., T. Yoshida et al, 2004 Int. Arch. Allergy Immunol, 133(3):239-247), IL-1β (Wesa and Galy 2001 Int. Immunol., 13(8):1053-61), imiquimod (Gupta et al, 2004 J. Cutan. Med. Surg. 38(5):338-52), epoxyvibsanin B (International Patent Publication No. WO 2002/060922) and others known in the art.

In still another embodiment, a biological sample subject to the assay described herein is obtained from a human subject that has been administered a composition containing anti-IL-12 antibody. For example, samples from subjects who have been administered anti-cytokine therapy for, e.g., Crohn's Disease, multiple sclerosis, atherosclerosis, and others. See, e.g., N. Engl. J. Med., 2004 351:2069-2079; and US Patent Publication No. 2006/0159655; Hauer et al, 2005 Circulation, 112(7):1054-1062), among others.

Yet a further class of biological samples subject to assay by the methods described here are biological fluids and tissues obtained from a human subject suffering from a condition characterized by the induction of IL-12 neutralizing antibodies. One such condition includes, without limitation, thymoma associated diseases, such as myasthenia gravis (see, e.g., H. Shiono et al, 2003 Internat. Immunol., 15(8):903-913; Meager et al, 1997 Lancet 350(9091):1596-1597.

### C. Sample Preparation

The assay protocol for measurement/detection of IL-12, as well as IL-12 neutralizing antibodies, involves obtaining the biological sample from a desirable source for testing as described above. Additionally, the sample may be processed or modified to remove inhibitory substances prior to testing. Among commonly known treatments are heat inactivation techniques, such as heating the sample to temperatures from room temperature to about 56° C. for less than about 30 minutes, followed by cooling. Still other heat inactivation methods are known to those of skill in the art.

The sample is optionally serially diluted by adding measured quantities of an inert solution, e.g., saline, buffered saline, a supplemented medium formulation or a suitable serum diluent, of which many are commercially available and known to one of skill in the art, and/or described in the examples below, to a desired quantity of sample.

When the assay is conducted to determine the presence or amount of IL-12 neutralizing antibodies, the sample is also pre-incubated with a known concentration or quantity of IL-12. The IL-12 is desirably human or mammalian IL-12, or active fragments thereof. IL-12 protein is generally available as a recombinant product from commercial sources, such as Genetics Institute, Cambridge, Mass. or Wyeth Biosciences, or may be generated recombinantly by known techniques. See, e.g., U.S. Pat. No. 5,648,467 and others known to those of skill in the art.

For pre-incubation with sample, in one embodiment, the IL-12 is added to the sample in concentrations that provide between about 0.1 to 10 IU/mL sample. In another embodiment, the pre-incubation concentration of IL-12 is between 3 to 6 IU/mL. In still a further embodiment, the pre-incubation concentration of IL-12 is 3 IU/mL. These ranges includes IL-12 concentrations that provide 0.1, 0.2, 0.3, 0.4, 0.5, 0.6, 0.7, 0.8, 0.9 IU/mL, including such incremental increases between concentrations providing 1, 2, 3, 4, 5, 6, 7, 8, 9 or 10.0 IU/mL of sample. The international unit is a more readily quantified amount because it is standardized. While a physical measurement of IL-12 concentration, such as ng/mL, could vary from lot to lot of IL-12, as a reference for general understanding, the IL-12 is best measured in IU. For ease of reference, 10 IU IL-12 is equivalent to about 1 ng of the IL-12 preparation used herein.

The pre-incubation of sample with the known amount or concentration of IL-12 occurs under conditions sufficient to allow the IL-12 to complex with any neutralizing anti-IL-12 antibody in the sample. For example, pre-incubation conditions comprise incubation from room temperature, i.e., 23° C. to about 37° C. for an incubation time of about 5 minutes to 24 hours. Thus, suitable pre-incubation temperatures include about, 23° C., 24° C., 25° C., 26° C., 27° C., 28° C., 29° C., 30° C., 31° C. 32° C., 33° C., 34° C., 35° C., 36° C., and about 37° C. Suitable pre-incubation times at these temperatures include from about 5 minutes, 10 minutes, 20 minutes, 30 minutes, 40 minutes, 50 minutes, about 1 hour, and similar increments up to 2, 3, 4, 5, 6, 7, 8, 9, 10, 11, 12, 13, 14, 15, 16, 17, 18, 19, 20, 21, 22, 23, or about 24 hours.

### D. Incubation with Cells

Once the sample is prepared, and optionally pre-incubated with a known amount of IL-12 for use in determining the neutralizing antibodies, the sample is incubated with a selected number of NK cells, e.g., NK-92MI cells, under conditions that allow the cells to secrete IFN-γ in response to IL-12.

In one embodiment, the NK cells, e.g., NK-92MI cells, are incubated with the sample at a cell number of between 15,000 to 400,000 cells/sample. In another embodiment, the NK cells, e.g., NK-92MI cells, are used at a cell number or volume of between 15,000 to 60,000 cells/sample. For example, the number of NK cells, e.g., NK-92MI cells, used in the assay includes about 15,000, or about 20,000 or about 30,000 or about 40,000 or about 50,000 or about 60,000 cells/sample. In still other embodiments, the cell number is about 75,000 or about 100,000 or about 150,000 or about 200,000 or about 250,000 or about 300,000 or about 350,000 or about 400,000 cells/sample.

In one embodiment, the incubation conditions comprise a temperature of about 37° C. in a 5% CO₂ incubator for between 6 to about 24 hours. Such a suitable incubation time period can include anywhere from about 6, 7, 8, 9, 10, 11, 12, 13, 14, 15, 16, 17, 18 19, 20, 21, 22, 23 up to 24 hours. This incubation period and conditions permit a measurable amount of IFN-γ to be secreted from the cells and maintains the viability of the cells. One of skill in the art may readily select from among other specific incubation conditions provided that these two latter requirements are met.

More preferably, this assay is performed by placing the samples in a conventional multi-well plate, such as a 96 well sample plate. Other conventional multi-well plates may be employed in the assay. Use of such plates enables an automated assay to be performed.

### E. Determination of Activation Factor/Cytokine Amounts

Following the above-described incubation period, activation factor/cytokine, e.g., IFN-γ, levels in the cell culture supernatants are measured by conventional assay techniques suitable for the measurement of the cytokine. As one example, a sandwich enzyme linked immunoassay is performed to measure the amount of IFN-γ secreted into each well. Such ELISAs are well known and may follow the instructions provided by the commercial sources of ELISA kits, such as the kit employed in the examples below, i.e., BD OptEIA™ ELISA kit (BD Biosciences, San Diego, Calif.). Still other ELISA methods and kits for determination of IFN-γ are known and available to one of skill in the art. This assay is not limited by the techniques employed to measure the secreted cytokine.

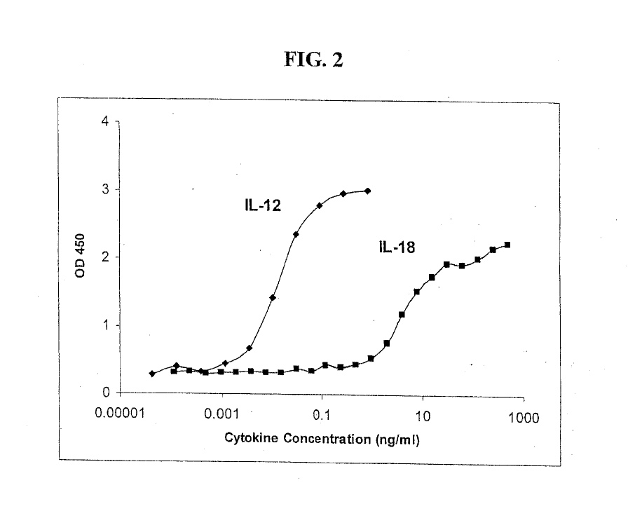

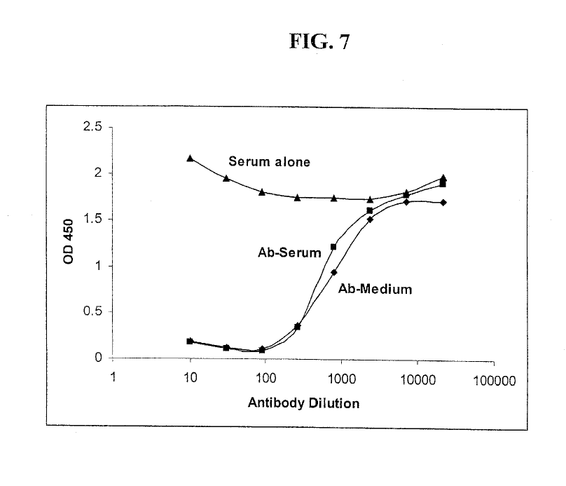

Because the secretion of IFN-γ by the NK-92MI cells is dependent on the dose of IL-12, the measurement of the IFN-γ level produced by the cells in this assay permits correlation to the presence and amount of IL-12 or anti-IL-12 neutralizing antibody in the sample by comparison generally to a reference standard curve. Reference standards of known IFN-γ concentration secreted in the same cells in response to known amount of IL-12 in the cell culture, i.e., typical dosage curves, are useful in the assay to assign IL-12 concentrations to unknown samples. In some embodiments, the reference is an optical density measurement (OD). FIGS. 2 and 7 are examples of such dosage curves. The reference standard may also be prepared during the same experiment by using a control cell which is exposed to a known amount of IL-12 and without the sample, but such controls are not necessary if a predetermined reference sample or curve is available.

### F. Other Embodiments of the Assay

This assay format may be varied in a manner desired to suit the particular situation in which IL-12 or anti-IL-12 antibodies are likely to be present in a sample.

For example, in another embodiment, a method for detecting human IL-12 in a human biological sample comprises incubating the NK cells, e.g., NK-92MI cells, or progeny thereof with a biological sample obtained from a human subject prior to administration to the subject of a composition comprising IL-12 and also incubating the cells with a sample obtained after administration of the composition comprising IL-12. Once the incubation step with the cells is performed, the levels of IFN-γ secreted by the cells in the pre-administration samples are compared with the levels of IFN-γ secreted by the cells in the post-administration samples. Performance of this biological assay can thus provide information on the effect of an immunogenic composition or vaccine or therapeutic containing IL-12 as principal component or as an adjuvant.

For example, in another similar embodiment, a method for detecting antibodies that neutralize the activity of human IL-12 in a human biological sample comprises incubating the NK cells, e.g., NK-92MI cells, or progeny thereof with a biological sample obtained from a human subject prior to administration to the subject of a composition comprising IL-12 and also incubating the cells with a sample obtained after administration of the composition comprising IL-12. Each of these biological samples is desirably pre-incubated with a known amount of IL-12, as provided in the assay description above. Once the incubation step with the cells is performed, the levels of IFN-γ secreted by the cells in the pre-administration samples are compared with the levels of IFN-γ secreted by the cells in the post-administration samples. The presence of neutralizing anti-IL12 antibodies in the post-administration samples is indicated by a reduction in the level of secreted IFN-γ as compared to the pre-administration sample IFN-γ level. Performance of this neutralization assay can thus provide information on the effect of an immunogenic composition or vaccine or therapeutic containing IL-12 as principal component or as an adjuvant.

In yet another embodiment, the method for detecting human Interleukin-12 in a human biological sample comprises preparing serial dilutions of the sample and incubating the dilutions according to the procedures described in detail above. To each dilution a known number of NK cells, e.g., NK-92 cells, NK-92MI cells or progeny thereof, are added and incubated for a time sufficient to allow the cells to secrete IFN-γ. The levels of IFN-γ secreted by the cells in each dilution are compared with a control IFN-γ level secreted by the NK cells, e.g., NK-92 cells, NK-92MI cells or progeny thereof, in the presence of a known amount of IL-12 (or to predetermined reference standards or curves), but without the sample. The use of serial dilutions in this assay format thereby permits a more detailed evaluation of the IL-12 produced. This may be desirable in situations in which the IL-12 concentration is greater than anticipated, or a more detailed quantification of IL-12 in the sample is indicated.

In yet another embodiment, the method for detecting antibodies that neutralize the activity of human Interleukin-12 in a human biological sample comprises adding a known amount of IL-12 to serial dilutions of the sample and incubating the dilutions according to the procedures described in detail above. To each dilution a known number of NK cells, e.g., NK-92 cells, NK-92MI cells or progeny thereof, are added and incubated for a time sufficient to allow the cells to secrete IFN-γ. The levels of IFN-γ secreted by the cells in each dilution are compared with a control IFN-γ level secreted by the NK cells, e.g., NK-92 cells, NK-92MI cells or progeny thereof, in the presence of a known amount of IL-12. The presence of neutralizing anti-IL12 antibodies in the serial dilutions is indicated by a reduction in the level of secreted IFN-γ in the serial dilutions as compared to the control IFN-γ level (or to the predetermined reference standard). The use of serial dilutions in this assay format thereby permits a more detailed evaluation of the antibodies produced. This may be desirable in situations in which the antibody production is greater than anticipated, or a more detailed quantification of antibody production is indicated.

In still yet further embodiments, the neutralization assay provided herein is used in a high-throughput, automated method for detecting IL-12 or antibodies that neutralize the activity of human IL-12 in a large number of samples. In a high-throughput method, a large number of human biological samples can be evaluated in one assay. Such a large sample number includes greater than 100 samples per assay performance. More specifically, the sample number may include greater than 50, 200, 300, 400, 500, 600, 700, 800, 900, 1000, 2000, 3000, 4000 or greater than 5000 samples per assay performance. This assay, like the formats previously described involves serially diluting a biological sample with culture medium in multiple wells on an assay plate. Robots may be used for sample preparation. When the assay format is used to detect the neutralizing antibodies, a known amount of IL-12 is introduced to each well and the wells are incubated for a time sufficient to allow the IL-12 to complex with any neutralizing IL-12 antibodies in the wells. To detect IL-12 in the samples, no additional IL-12 is added. Then, to each well is then added a known number of NK-92MI cells or progeny thereof. The wells are incubated for a time sufficient to allow the cells to secrete IFN-γ. The levels of IFN-γ secreted by the cells into each well is then measured, e.g., by sandwich ELISA, and the levels are compared with control IFN-γ levels secreted by the NK-92MI cells in the presence of a known amount of IL-12 or a set of predetermined reference standards. As with the above description of the assays, the presence of IL-12 in the wells is measured by a variation from the reference standard or by location on a reference curve. The presence of neutralizing anti-IL12 antibodies in the wells is indicated by a reduction in the level of secreted IFN-γ in the wells as compared to a control IFN-γ level.

### G. Advantages of the Assay

The assays described herein have several advantages over other assay formats for the measurement of IL-12 or anti-IL-12 neutralizing antibodies. For example, the use of a stable cell line gives the assay consistency and reproducibility of the biological response. As well, the 96-well format gives the assay high throughput capability. Higher throughput capability is beneficial because the number of samples that can be processed often limits biological assays, such as neutralization assays. For determining antibodies to cytokines, a high throughput neutralization assay is especially valuable. Having a reliable neutralization assay avoids the occurrence of non-specificity in other assay types and still provides information on the existence of biologically active antibodies, if any, in serum samples.

Another advantage of this assay is that by basing the assay on IFN-γ secretion, an accurate and efficient determination of IL-12 concentration or anti-IL-12 neutralization antibody can be completed within 24 hours. In contrast, other biological assays dependent on cell proliferation require several days to complete. The rapidity with which this IL-12 neutralization assay can be performed increases the number of runs that can be performed on a weekly basis. Furthermore, if an alternative cellular component is measured that is responsive to IL-12, but precedes IFN-γ secretion, the time to complete the assay may be further shortened.

Thus this assay format is efficient for the rapid determination of antibody generation in subjects being administered compositions that induce such antibodies. As demonstrated by the examples below, the specificity of this assay for anti-IL-12 neutralizing antibodies has been confirmed. Testing of commercial human IgG preparations and several normal human sera failed to detect neutralizing antibodies to IL-12. Coupled with the development of culture methods based on such stable cell lines, and other reagents applicable to higher throughput formats, such biological assays can be used for larger numbers of samples. It has further been observed that the human sera tested to date displayed low background and do not appear to have interfering components. Therefore, any antibodies generated to IL-12 should be readily measured by this assay. This assay can be used in non-human primates and has been employed to test post-vaccination samples in human clinical trials and experimental evaluations in Rhesus macaques in which the vaccines were formulated with an IL-12 adjuvant.

### H. Uses of the Assay

In one aspect, the assay to measure IL-12 neutralizing antibodies permits one to determine if neutralizing antibodies are generated to IL-12 when delivered as a vaccine adjuvant or therapeutic treatment, as described above, or to detect unmetabolized IL-12 from such vaccines or therapeutic treatments. Although responsiveness of cells to IL-12 has been detailed in the literature, a neutralization assay designed to screen human serum has as yet not been described. As noted above, IL-12 neutralizing antibodies can arise in myasthenia gravis patients with thymomas (Meager, A., et al, 2003, Clin. Exp. Immunol. 132:128-36; Zhang W., et al, 2003 J. Neuroimmunol., 139:102-8). As well, therapeutic treatment with IL-12 has in some instances induced antibodies to IL-12 (Atkins, M. B., et al, 1997 Clin. Cancer Res., 3:409-17). Screening of many human sera using the assay described herein indicates that neutralizing antibodies to IL-12 in the general population are rare. Investigations designed to therapeutically modulate IL-12 activity either by increasing or reducing cytokine levels are being developed. Thus, an assay such as the neutralizing IL-12 antibody assay described herein finds applicability in clinical situations wherein IL-12 enhancement or diminution is involved.

Other uses of the IL-12 bioassay or neutralization assay involve conditions in which antibodies to IL-12, or IL-12 itself, are important for human health, or where a specific therapy induces these types of antibodies. As demonstrated by the documents cited above, such therapy involves delivery of anti-IL-12 drugs (antibodies) to help reduce disease caused by overactive inflammatory responses, or IL-12 inducing compounds including IL-12 itself to improve cellular immune responses.

At present, the IL-12 neutralization assay described herein is able to test for any IL-12 specific antibodies that are generated when IL-12 is used as an adjuvant in vaccine formulations or as a primary therapeutic treatment. The methods are also useful as a safety test for vaccines that contain IL-12. This neutralization assay to quantify IL-12 neutralizing antibodies in human serum uses a cell line and reagents compatible with a 96-well format. Thus the assay has sufficient throughput that it may be used for primary testing of clinical samples.

In order that this invention may be better understood, the following examples are set forth. The data from the examples below indicates that the IL-12 neutralization assay described herein has the capability of measuring IL-12 neutralizing antibodies in mammalian, including human, serum. The examples are for the purpose of illustration only and are not to be construed as limiting the scope of the invention. All documents, publications and patents cited in the following examples and throughout this specification are incorporated by reference herein.

### EXAMPLE 1

### Cells and Reagents

NK-92MI cells were purchased from the American Type Culture Collection, Manassas, Va. (ATCC Catalog No: CRL-2408, Lot 2634959) and passaged in Growth Medium (Alpha minimal medium, no ribose or deoxyribose; Mediatech, Herndon, Va.) supplemented with 12.5% Fetal bovine serum (Invitrogen, Grand Island, N.Y.), 12.5% Horse Serum (Sigma, St. Louis, Mo.), 2 mM L-glutamine (Invitrogen), 1× antibiotic/antimycotic (Invitrogen), 1.5 gm/L sodium bicarbonate (Mediatech), 0.2 mM Inositol (Sigma), 0.1 mM 2-mercaptoethanol (Sigma), and 0.02 mM folic acid (Sigma)).

Recombinant human IL-12 was generated by Wyeth BioPharma (Lot 4A18I006), and recombinant human IL-18 was purchased from R&D Systems (Minneapolis, Minn.). Recombinant rhesus IL-12 was generated by Wyeth Vaccines Discovery by in vitro expression of plasmid encoded rhesus IL-12. The recombinant IL-12 used for this assay was compared with the international standard for IL-12 from the National Institute for Biological Standards and Controls (NIBSC) and was found to have a biological activity of 10 international units (IU) per ng.

Purified goat polyclonal antibodies against human IL-12 were purchased from R&D Systems and PeproTech (Rocky Hill, N.J.). The commercial antibodies were diluted into a pool of human serum negative in the neutralization assay to equalize the volume of human serum in the sample and control samples.

Human sera for testing or to generate a serum pool were purchased from either Bioreclamation Inc. (Hicksville, N.Y.), or Intergen, (Norcross, Ga.).

Assay Medium used was RPMI 1640 with glutamine (Mediatech) supplemented with 10% Fetal Bovine serum.

Reagents for the IFN-γ ELISA were purchased as part of a BD OptEIA™ ELISA kit (BD Biosciences, San Diego, Calif.). The Immunolon™ 2HB plates (ThermoLab Systems, Franklin, Mass.) were used as the 96-well ELISA plates.

### EXAMPLE 2

### IFN-γ Enzyme Linked Immunosorbent Assay

Using the procedures outlined by the BD OptEIA™ ELISA kit, plates were coated overnight at 4° C. with the anti-IFN-γ capture antibody (1:250 in PBS). Plates were washed 3 times and blocked with PBS +1% BSA for 1-3 hrs at room temperature. Following 3 washes, 50 μL of assay medium was added to each well on the plate and supernatants (50 μl) from the cell culture plate were then transferred onto the ELISA plate. Following 1 hour incubation at room temperature the plates were washed 3 times and secondary antibody-enzyme conjugate was added and incubated 1 hour at room temperature. The plates were then washed 6 times and substrate was added and the reaction allowed to proceed for approximately 8 minutes before being stopped and the color read in a plate reader using a wavelength of 450 nm. The intensity of the color provided the indication of IFN-γ levels secreted by the cells.

### EXAMPLE 3

### Characterization of NK-92MI Cells

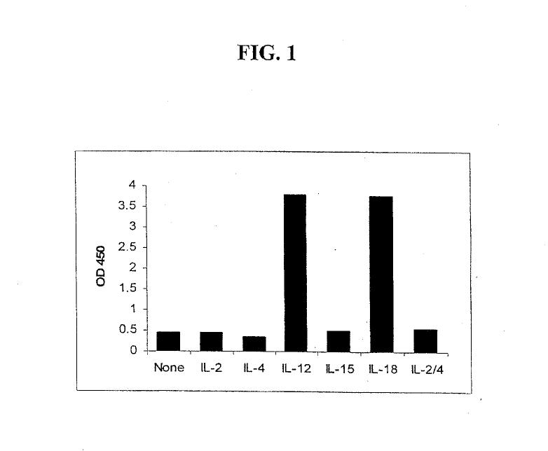

The response of the NK-92MI cell line to various cytokines was evaluated under conditions of high cell numbers (400,000 per well) and large doses (10 ng/mL) of cytokines. The NK-92MI cells were cultured in the presence of 10 ng/mL of one of a negative control, IL-2, IL-4, IL-12, IL-15, IL-18 or an IL-2/4 fusion protein. Performance of the IFN-γ ELISA of Example 2, demonstrated that both IL-12 and IL-18 were able to induce the secretion of significant amounts of IFN-γ from the NK-92MI cells. See FIG. 1.

These two latter cytokines were tested again under conditions of lower cell numbers (30,000/well) and a range of cytokine concentrations from about 0.0001 through 1000 ng/mL. Performance of the same ELISA of Example 2 determined that the cells responded to lower doses of IL-12 than IL-18 (FIG. 2). The levels of IFN-γ that can be detected by the IFN-γ ELISA are readily recovered from culture supernatants when standard assay conditions are used.

### EXAMPLE 4

### Neutralization Assay Using NK-92MI Cells

A. Assay Protocol

The neutralization assay described herein was performed using the following protocol:

Serum and control samples were serially diluted (usually 2-fold) in a 96 well plate (Costar Flat bottom, Coming, N.Y.). Assay medium was used to perform the dilutions. To the diluted samples, an equal volume of Assay Medium containing 12 IU/mL (1.2 ng/mL of IL-12) was added and incubated for 1 hr at room temperature to allow the antibodies in the serum to combine with the IL-12.

Cultured NK-92MI cells were spun down in 50 mL centrifuge tubes at 250×g for 10 min, washed once in Assay Medium, counted and suspended at 3×10⁵ cells/mL. The cells were then added to the plate containing the serum/IL-12 mixture in a volume equal to that already present in the wells on the plate. The plate was then incubated overnight at 37° C. in a 5% CO₂ incubator.

B. Assay Performance

Based on the IL-12 dose curve (FIG. 2), a concentration of 3 IU/mL 1 (or 0.3 ng/mL) chosen to stimulate the NK-92MI cells in this neutralization assay. This dose produced maximal effect, but does not represent a large excess of cytokine that would reduce the sensitivity of the assay. The two commercially prepared antibodies specific for human IL-12 of Example 1 were tested for their ability to neutralize IL-12 biological activity in this assay. Antibody preparations were serially diluted in a 96 well-pate prior to the addition of IL-12 to the culture wells. Following a 1 hour incubation of antibody/cytokine, 30,000 NK-92MI cells were added to each well. IFN-γ levels in overnight cell culture supernatants were measured by the ELISA of Example 2.

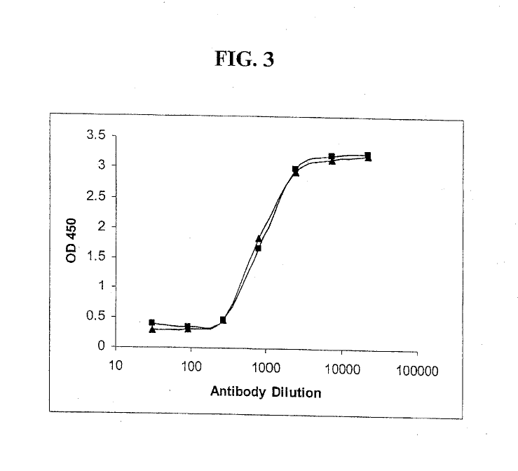

As demonstrated in FIG. 3, high concentrations of antibody were able to cause full inhibition of IFN-γ secretion from the NK-92MI cells, as would be expected for antibodies that neutralize the IL-12 added to the culture. As the antibody was diluted, and more IL-12 became available, the amount of IFN-γ secreted by the cells increased. These data indicated that the presence of an IL-12 specific neutralizing antibody was detectable by this assay.

### EXAMPLE 5

### Specificity of Neutralization Assay

To examine the specificity of the IL-12 Neutralization Assay using NK-92MI cells, commercially prepared antibodies to human cytokines, i.e., human IL-2, IL-3, IL-4, IL-5, IL-15, IL-18, GM-CSF and M-CSF, were each tested in the assay of Example 4 (data not shown). No effect on IFN-γ secretion was found when these other cytokine specific antibodies were added alone, or to an IL-12 positive control, indicating that antibodies to these other cytokines do not interfere in the assay.

To determine if IL-12 specific antibodies may actually interfere with IFN-γ secretion from NK-92MI cells by a method other than the neutralization of IL-12, the IL-12 specific antibodies were tested in a non-IL-12 dependent assay. Negative serum pool (NSP) alone or with the addition of anti-IL-12 antibodies or anti-IL-18 antibodies were serially diluted in a 96-well plate prior to the addition of cytokine to culture wells. Following a 1 hr incubation of antibody/cytokine, 60,000 NK-92MI cells were added to each well. NK-92MI cells were stimulated with IL-18 (rather than IL-12) to secrete IFN-γ and then the effect of the anti-IL-12 antibodies was tested. The final IL-18 concentration after addition of the NK-92MI cells was 30 ng/mL. IFN-γ levels in overnight cell culture supernatants were measured by an ELISA in optical density at 450 nm wavelength.

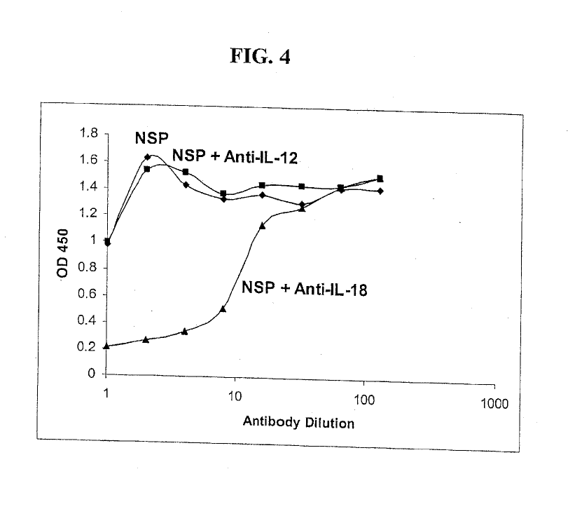

As shown in FIG. 4, when antibodies to IL-12 and IL-18 were diluted into the negative serum pool, only antibodies to IL-18 inhibited IFN-γ secretion. This indicated that the activity of IL-12 antibodies was through the neutralization of the IL-12 and not due to some toxic effect on the cells or interference with another aspect of the assay.

### EXAMPLE 6

### Lack of IL-12 Neutralizing Antibody in Normal Human Sera

The IL-12 neutralization assay of Example 4 has been used to test more than 30 individual human sera, a large pool of different human sera, and several types of prescription Immune Globulin G preparations also made from multiple donors.

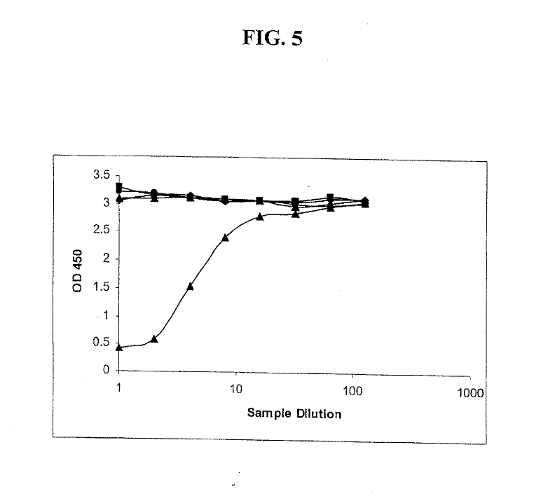

FIG. 5 shows typical curves for a commercial goat anti-IL-12 standard antibody and four individual human sera. Because no source of human antibody specific for IL-12 is available, the assay was standardized using antibody prepared in goats. Although differences in the avidity of human antibodies compared to the goat polyclonal serum may occur, this should not greatly affect the ability to detect human antibodies. As indicated in FIG. 5, no neutralizing activity (maximal IFN-γ secretion) was demonstrated by the human sera, but a dose dependent neutralization was found when using the commercial IL-12 specific antibody. No IL-12 neutralizing activity has been found in any normal human sample tested to date. Many of these same samples had GM-CSF neutralization activity (data not shown), indicating that unlike antibodies to GM-CSF that are known to occur in a small percent of the population, neutralizing antibodies to IL-12 may be rare.

### EXAMPLE 7

### IL-12 Neutralization Assay in a Non-Human Primate

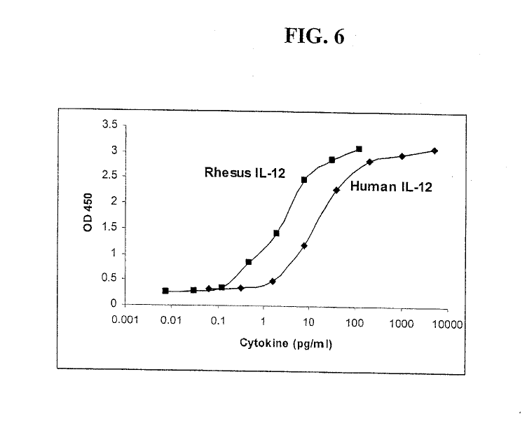

Experiments were performed according to the neutralization assay of Example 4 to determine if the NK-92MI cells (30,000 cells/well) could be used to test samples from Rhesus macaques. Recombinant Rhesus IL-12 was tested for its ability to induce NK-92MI cells to secrete IFN-γ, measured in OD₄₅₀. FIG. 6 shows a comparison of the dose curves for varying doses (pg/mL) of Rhesus IL-12 and Human IL-12. The Rhesus IL-12 was effective at the induction of IFN-γ secretion indicating that NK-92MI cells recognize Rhesus IL-12.

Using a 0.03 ng/mL dose of Rhesus IL-12 to stimulate NK-92MI cells (30,000 cells/well), the neutralization assay of Example 4 was performed using Rhesus serum alone, Rhesus serum spiked with the commercially prepared anti-human IL-12 antibody (R&D Systems, Minneapolis, Minn.), or the human anti-IL-12 antibody in medium. The antibody preparations were first diluted to 1:10 in either medium, or Rhesus serum, and then serially diluted in a 96-well plate prior to the addition of Rhesus IL-12 to culture wells. As well, a third test sample was Rhesus serum alone with no added IL-12 antibody. No antibody specific for Rhesus IL-12 was available for these studies. Following a 1 hr incubation of antibody/cytokine, 30,000 NK-92MI cells were added to each well. The final Rhesus IL-12 concentration after addition of the NK-92MI cells was 0.03 ng/mL. IFN-γ levels in overnight cell culture supernatants were measured by an ELISA in optical density at 450 nm wavelength.

The results are shown in FIG. 7. The Rhesus serum alone did not show any inhibition of IFN-γ secretion, indicating that no anti-IL-12 activity was present in the serum. Adding the human anti-IL-12 antibody to the Rhesus serum was able to inhibit IFN-γ secretion, indicating that the human specific antibody did recognize the rhesus IL-12. Further, this data verified that the Rhesus based assay detected an IL-12 neutralizing antibody in serum. The neutralization curves of the anti-IL-12 antibody diluted in Rhesus serum, or the anti-IL-12 antibody diluted in medium were similar, indicating that the Rhesus serum did not contain a component that significantly impaired the measurement of IL-12 neutralizing antibody.

The experiments performed with the Rhesus IL-12 indicated that the IL-12 neutralization assay using the NK-92MI cell line could be used to test for neutralizing antibodies specific for Rhesus IL-12 in serum, or to test for Rhesus IL-12 itself.

### EXAMPLE 8

### Evaluation of IL-12 Neutralizing Antibody Following Vaccination With IL-12 Adjuvanted Vaccines

Samples from a human vaccine clinical trial and a vaccination trial in Rhesus macaques that used IL-12 as an adjuvant have been tested for neutralizing antibodies to IL-12 using the IL-12 neutralization assay of Example 4.

The neutralization assay described herein was employed to assess the effect of IL12 adjuvants on candidate plasmid DNA (pDNA) vaccine designs. In this study, a number of 2-, 3- and 4-vector pDNA vaccine designs encoding HIV env, gag, pol, nef, tat and vif were tested for their ability to elicit HIV-1 antigen-specific CMI and humoral immune responses in rhesus macaques. These vaccine compositions were adjuvanted by IL-12 as set out in Table 1. The results of this study indicate that all of 2 and 3 vector vaccine designs were capable of eliciting measurable total HIV-specific CMI responses. The resulting observations of the neutralization assay, as confirmed by an ELISA, indicated that no neutralizing IL-12 antibody interfered with the vaccine efficacy.

For this study a total of 36, Mamu-A*01 negative, captive-bred, male rhesus macaques (macaca mulatta) of Indian origin were used. Macaques were maintained in accordance with the Guide for the Care and Use of Laboratory Animals (National Research Council, National Academic Press, Washington, D.C., 1996).

Briefly described, plasmid DNAs used in these experiments employed HIV gag p55 (HXB2), pol (HXB2), nef (NL4-3), tat (NL4-3), vif (NL4-3) and env gp160 (primary isolate 6101 sequences, which were RNA optimized. Expression cassettes encoding HIV-1 derived genes were constructed using one of the following two transcriptional unit designs: (i) human cytomegalovirus (HCMV) immediate early promoter and bovine growth hormone (BGH) polyadenylation (polyA) signal; (ii) simian cytomegalovirus (SCMV) promoter and BGH polyA. In addition, expression vectors included a chimeric kanamycin resistance (kmr) gene, adenylyl 4′-nucleotidyl transferase type 1a (Shaw, K.J., et al Microbiological Reviews, 1993. 57(1): p. 138-63 ; Sadaie, Y., et al J. Bacteriol. 1980. 141(3): p. 1178-82) and a ColE1 bacterial origin of replication required for selection and propagation of bacteria, respectively.

An expression plasmid encoding rhesus IL-12 was co-delivered as a molecular adjuvant. The dual-promoter IL-12 expression vector encodes the rhesus IL-12 p35 and p40 genes. The IL-12 p35 subunit is expressed under control of the HCMV immediate early promoter and SV40 polyA signal, while the IL-12 p40 subunit is expressed under control of the SCMV and BGH polyA signal. Production of rhesus IL-12 was confirmed after transient transfection of RD cells and screening cell supernatants for cytokine expression using an anti-human IL-12 p70 capture ELISA (data not shown; Endogen, Woburn, Mass.).

The neutralization assay described in Example 4 herein was used to assess the bioactivity of the plasmid-expressed rhesus IL-12 by assaying supernatants from transiently transfected RD cells for the capacity to induce IFN-γ secretion in resting rhesus peripheral blood lymphocytes (PBLs; data not shown).

The vaccination protocol was as follows: Immediately prior to inoculation, the appropriate pDNA expression vectors were mixed. A fixed total dose of vaccine pDNA (8.5 mg, groups 2d, 3a, 3c, 4a) was administered in combination with 1.5 mg of the dual promoter pDNA encoding rhesus IL-12 p40 and p35 by intramuscular injection into both deltoid and quadriceps muscles (1.0 cc/injection site, 2.5 mg DNA per site) using an 18-gauge needle and 3 mL syringe. Control macaques were primed with 8.5 mg of empty expression vector DNA in combination with 1.5 mg plasmid IL-12. For macaques receiving the pDNA vaccine in combination with in vivo electroporation (EP; i.m. injections and in vivo electroporations were performed using a combined injection/electroporation device where the injection needles also served as electrodes; group 3cE), one-fifth of the total vaccine dose (1.7 mg pDNA vaccine in combination with 0.3 mg IL-12 pDNA) was used. The administrations were conducted on a schedule of 0, 4, and 8 weeks.

The combinations of pDNA-based expression vectors encoding one or two unique expression cassettes are set out in Table 1. As used in the table, note (a) refers to the human cytomegalovirus immediate early promoter; note (b) refers to a fusion of the HIV-1 HXB2 gag-pol gene. Note (c) refers to the simian cytomegalovirus immediate early promoter. Note (d) refers to the HIV-1 6101 env gp160. Note (e) refers to a fusion of the HIV-1 NL43 nef, tat and vif genes.

TABLE 1

| | No. | Vector Dose | |
|---|---|---|---|
| Group # | animals | (mg) | Encoded antigens in Vector |
| 2d | 6 | 4.25 | HCMV^{a}-gag/pol^{b} |
| | | 4.25 | SCMV^{c}-env^{d}; HCMV-ntv^{e} |
| | | 1.5 | HCMV-IL-12 p35/SCMV-IL-12 p40 |
| 3a | 6 | 2.8 | HCMV-gag/pol |
| | | 2.8 | HCMV-env |
| | | 2.8 | HCMV-ntv |
| | | 1.5 | HCMV-IL-12 p35/SCMV-IL-12 p40 |
| 3c | 6 | 2.8 | SCMV-env; HCMV-ntv |
| | | 2.8 | HCMV-gag |
| | | 2.8 | HCMV-pol |
| | | 1.5 | HCMV-IL-12 p35/SCMV-IL-12 p40 |
| 3cE | 6 | 0.56 | SCMV-env; HCMV-ntv |
| | | 0.56 | HCMV-gag |
| | | 0.56 | HCMV-pol |
| | | 0.56 | HCMV-IL-12 p35/SCMV-IL-12 p40 |
| 4a | 6 | 2.13 | HCMV-gag |
| | | 2.13 | HCMV-env |
| | | 2.13 | HCMV-pol |
| | | 2.13 | HCMV-ntv |
| | | 1.5 | HCMV-IL-12 p35/SCMV-IL-12 p40 |
| Con | 6 | 8.5 | Empty vector control |
| | | 1.5 | HCMV-IL-12 p35/SCMV-IL-12 p40 |

Macaques were assessed by daily observations and by weekly physical examinations, body weight measurements and for changes in peripheral blood cell subsets by complete blood count (CBC) analysis. Over the course of the vaccination regimen, all macaques appeared normal and had body weights which were stable or increased during the study interval. Macaques immunized with the candidate pDNA vaccines by i.m. injection using a standard needle and syringe maintained stable peripheral blood bulk cell subsets (i.e. total white blood cell, total lymphocyte, CD20⁺ cell counts) as determined by CBC analysis (data not shown).

While antigen specific antibody titers for the HIV peptides were assayed by an IFN-γ ELISpot assay, the rhesus IL-12-binding and neutralizing antibody were evaluated using a commercially available human IL-12 ELISA kit (Pierce Endogen) to detect anti-IL-12 antibody in serum and the neutralizing assay of Example 4.

First the ELISA was performed as follows: Microplate strips pre-coated with anti-human IL-12 antibody were incubated with 20 ng/well of recombinant human IL-12 for 2 hr at 37° C. The IL-12 loaded strips were then washed and incubated with test serum serially diluted with 1×PBS containing 1% BSA and 0.1% Tween®-20 reagent (1% BSA/PBST) overnight at 4° C. A biotin conjugated primary antibody specific for monkey IgG diluted in 1% BSA/1×PBS at 1:30,000 (Accurate Scientific, Westbury N.Y.), followed by a streptavidin-horseradish peroxidase conjugated anti-biotin antibody (500 units/mL stock, diluted 1:10,000 with 1×PBS supplemented with 0.1% Tween®-20 reagent, 1% BSA, Roche Immunochemical, Indianapolis, Ind.) was added to the plates. The strips were then developed with TMB (Sigma) and O.D. was read at 450 nm. Anti-rhesus IL-12 antibody titers were defined as the reciprocal of the last serum dilution giving an O.D.₄₅₀ greater than the same macaques's naïve serum (i.e. week 0) +3 standard deviations.

Serum samples containing anti-rhesus IL-12 antibodies as detected by the ELISA were further screened in the Neutralizing Assay of Example 4, i.e., to determine if the antibodies possessed the ability to block the proliferation of the IL-12 responsive cell line, NK-92MI. Briefly, test serum was diluted 1:10 in growth medium (Alpha Minimal medium no ribose or deoxyribose, (Mediatech, Herndon, Va.) supplemented with 12.5% FBS (Invitrogen, Grand Island, N.Y.), 12.5% Horse Serum (Sigma, St. Louis, Mo.) 2 mM L-glutamine (Invitrogen), 1× antibiotic/antimycotic (Invitrogen), 1.5 g/L sodium bicarbonate (Mediatech), 0.2 mM Inositol (Sigma), 0.1 mM 2-mercaptoethanol (Sigma), and 0.02 mM folic acid (Sigma)) and then serially diluted 1:3 in a 96 well plate. To the diluted serum samples an equal volume (50 μL) of assay medium containing 0.12 ng/mL rhesus IL-12 was added and incubated for 1 hr at room temperature. NK-92MI cells (ATCC, Catalog No: CRL-2408, Lot 2634959, Manassas, Va.) were then added in 100 μL volume to each well of the 96 well plate containing the serum/IL-12 mixture and incubated overnight at 37° C. in a 5% CO₂ incubator. IL-12 dependant secretion of IFN-γ into the culture supernatant was quantified using a commercially available IFN-γ ELISA kit (BD Biosciences, San Diego, Calif.) as per the manufacturer's instructions.

ANOVA analysis of IFN-γ ELISpot data was performed on SAS version 8.2 software, using the type 1 GENMOD procedure with negative binomial distribution. Log₁₀ transformed ELISA and Neutralizing antibody data were analyzed using the SAS version 8.2 software, the least square means of each group were compared using the Mixed procedure. In all cases, p-values less than 0.05 indicated that the tests were statistically significant.

The results of these assays were as follows: Two weeks after the final pDNA immunization, peripheral blood lymphocytes (PBLs) were collected from the immunized macaques and screened for the presence of HIV-1 antigen-specific CMI responses by IFN-γ ELISpot analysis. In addition, the frequency by which the various vaccine designs were capable of eliciting HIV env-, gag-, pol-, nef, tat- and vif-specific responses in individual macaques within a given group were tabulated. The three vector vaccine design represented by group 3a of Table 1 elicited the greatest total HIV-specific IFN-γ ELISpot response with vaccine designs represented by groups 3c and 2d eliciting somewhat lower total HIV-specific IFN-γ ELISpot responses. However, the reduced responses elicited by the pDNA vaccine designs represented by 2d and 3c were not significantly different relative to group 3a. The lowest total HIV-specific IFN-γ ELISpot response was seen in the group receiving the four vector vaccine design represented by group 4a. The relative ranking of the various pDNA vaccine designs in terms of their ability to elicit antigen-specific IFN-γ ELISpot responses (i.e. 3a>2d=3c>4a) remained consistent regardless of whether the IFN-γ ELISpot responses were measured after the first, second, or third immunization (data not shown).

Immunized rhesus macaques that were also monitored for the induction of potentially autoreactive anti-IL-12 serum antibodies by ELISA. In Table 2 below, results are provided with antibody endpoint titers reported as the highest reciprocal dilution of serum giving an OD 450 reading 3-fold higher than week 0 (pre-immune) samples.

TABLE 2

| Group # | Macaque ID | Week 6 | Week 10 | Week 16 |
|---|---|---|---|---|
| 2d | A1N093 | — | — | — |
| | A1N098 | — | — | — |
| | A1N111 | — | — | — |
| | A2N040 | — | — | — |
| | A2N043 | — | — | — |
| | A2N065 | — | — | — |
| 3a | A1N020 | 450 | 50 | 450 |
| | A1N104 | — | — | — |
| | A2N009 | — | — | — |
| | A2N024 | — | — | 450 |
| | A2N027 | — | — | — |
| | A2N028 | — | — | — |
| 3c | A1N089 | — | — | — |
| | A1N197 | — | — | — |
| | A2N029 | — | — | — |
| | A2N042 | — | — | — |
| | A2N060 | — | — | — |
| | A2N070 | — | — | — |
| 3cE | A1N078 | — | — | — |
| | A1N086 | — | — | — |
| | A2N010 | — | — | — |
| | A2N014 | — | — | — |
| | A2N036 | — | — | — |
| | A2N054 | — | — | — |
| 4a | A1N092 | — | — | — |
| | A1N053 | — | — | — |
| | A2N077 | — | — | — |
| | A2N093 | — | — | — |
| | A2N088 | — | — | — |
| | A2N094 | — | — | — |
| Con | A1N040 | — | — | — |
| | A1N080 | — | — | — |
| | A1N112 | 450 | — | — |
| | A1N118 | — | — | — |
| | A2N037 | — | — | — |
| | A2N049 | — | — | — |

As seen in the Table 2, significantly elevated serum anti-IL-12 IgG antibody responses, relative to pre-immune serum samples, were routinely detected in only a single immunized macaque. Sporadic serum anti-IL-12 IgG responses were detected two other macaques. Importantly, these low level anti-Il-12 serum antibodies were found to be non-neutralizing in that they were unable to block the ability of exogenous IL-12 protein to stimulate the IL-12 responsive NK-92MI cell line to secrete detectable IFN-γ (data not shown).

Even though no test samples have been found to contain IL-12 neutralizing antibodies, the performance of the assay and its controls indicated that the assay was effective in its intended purpose to screen serum samples for neutralizing antibodies to IL-12. Thus, the neutralizing assay described is useful is determining the biological activity of anti-IL-12 antibodies produced to the small amount of IL-12 administered as a vaccine adjuvant.

All documents and publications cited herein are incorporated by reference. Various modifications and minor alterations in the method and components are believed to be clear to those of skill in the art.

What is claimed is:

1. A method for detecting antibodies that neutralize the activity of human Interleukin-12 (IL-12) in a human biological sample comprising:
(a) incubating a Natural Killer (NK) cell line or progeny thereof with said sample that has been pre-incubated with IL-12; and
(b) comparing the levels of IFN-γ secreted by said cells in said sample with a control IFN-γ level secreted by the same NK cells or progeny thereof in the presence of a known amount of IL-12;
whereby the presence of neutralizing anti-IL12 antibodies in said sample is indicated by a reduction in the level of secreted IFN-γ in said sample as compared to the control IFN-γ level.

2. The method according to claim 1, wherein said NK cells are NK-92 cells (ATCC Accession No. CRL-2407) or progeny thereof.

3. The method according to claim 1, wherein said NK cells are NK-92MI cells (ATCC Accession No. CRL-2408) or progeny thereof.

4. The method according to claim 1, wherein said method is completed within 24 hours.

5. The method according to claim 1, wherein said incubating occurs under conditions that allow said cells to produce IFN-γ in response to IL-12.

6. The method according to claim 5, wherein said incubation conditions comprise 37° C. in a 5% CO₂ incubator for between 6 to 24 hours.

7. The method according to claim 1, wherein said step (a) comprises incubating said NK cells at a cell number or volume of between 15,000 to 400,000 cells/sample.

8. The method according to claim 1, further comprising serially diluting said sample prior to pre-incubation.

9. The method according to claim 1, wherein said pre-incubation amount of IL-12 is between 0.1 to 10 IU/mL.

10. The method according to claim 1, wherein said pre-incubation occurs under conditions sufficient to allow said IL-12 to complex with any neutralizing IL-12 antibody in said samples.

11. The method according to claim 10, wherein said pre-incubation conditions comprise incubation from room temperature to about 37° C. for about 5 minutes to 24 hours.

12. The method according to claim 1, wherein said comparing comprises performing an enzyme linked immunoassay to measure said IFN-γ.

13. The method according to claim 1, wherein said comparing comprises a quantitative measurement of a level of said IL-12 neutralizing antibody based upon the IFN-γ level.

14. The method according to claim 1, further comprising placing said sample in a 96 well sample plate.

15. The method according to claim 1, further comprising obtaining said biological sample from a human subject that has been administered a composition containing IL-12 or that has been administered a composition expressing IL-12.

16. The method according to claim 1, further comprising obtaining said biological sample from a human subject that has been administered a composition expressing IL-12 selected from a group consisting of DNA plasmid, a viral vector, attenuated viral vector and viral replicon.

17. The method according to claim 16, wherein said composition expression IL-12 is a DNA plasmid.

18. The method according to claim 1, further comprising obtaining said biological sample from a human subject that has been administered a composition containing an IL-12-inducing agent.

19. The method according to claim 1, further comprising obtaining said biological sample from a human subject suffering from a condition characterized by the induction of IL-12 neutralizing antibodies.

20. The method according to claim 19, wherein said condition is myasthenia gravis.

21. The method according to claim 1, wherein said control is a predetermined reference standard or reference dosage curve.

22. A method for measuring the presence or amount of human IL-12 in a biological sample comprising incubating a Natural Killer (NK) cell line or progeny thereof with said sample; measuring the levels of IFN-γ secreted by said cells in said sample; and comparing said levels with a reference standard dosage curve.

23. The method according to claim 22, wherein said reference standard is the amount of IFN-γ level secreted by the same NK cells or progeny thereof in the presence of known amounts of IL-12.

24. A method for detecting antibodies that neutralize the activity of human Interleukin-12 (IL-12) in a human biological sample comprising:
(a) incubating a Natural Killer (NK) cell line or progeny thereof with a sample from a human subject prior to, and after, administration to said subject of a composition comprising IL-12, wherein each said sample is pre-incubated with IL-12; and
(b) comparing the levels of IFN-γ secreted by said cells in said pre-administration samples with the levels of IFN-γ secreted by said cells in said post-administration samples,
whereby the presence of neutralizing anti-IL12 antibodies in said post-administration samples is indicated by a reduction in the level of secreted IFN-γ as compared to the pre-administration sample IFN-γ level.

25. A method for detecting antibodies that neutralize the activity of human Interleukin-12 (IL-12) in a human biological sample comprising:
(a) adding a known amount of IL-12 to dilutions of said sample and incubating said dilutions;
(b) adding to each dilution a known number of a Natural Killer (NK) cell line or progeny thereof and incubating said dilutions for a time sufficient to allow said cells to secrete IFN-γ;
(c) comparing the levels of IFN-γ secreted by said cells in each dilution with a control IFN-γ level secreted by said NK cells in the presence of a known amount of IL-12 and without said sample;
whereby the presence of neutralizing anti-IL12 antibodies in said serial dilutions is indicated by a reduction in the level of secreted IFN-γ in said serial dilutions as compared to said control IFN-γ level.

26. A high-throughput, automated method for detecting antibodies that neutralize the activity of human Interleukin-12 (IL-12) in a large number of human biological samples comprising:
(a) diluting a biological sample with culture medium in multiple wells on an assay plate;
(b) adding a known amount of IL-12 to each well and incubating said wells for a time sufficient to allow said IL-12 to complex with any neutralizing IL-12 antibodies in said wells;
(c) adding to each well a known number of a Natural Killer (NK) cell line or progeny thereof and incubating said wells for a time sufficient to allow said cells to secrete IFN-γ;
(d) measuring the levels of IFN-γ secreted by said cells in each well;
(e) comparing said IFN-γ level in each well with a control IFN-γ level secreted by said NK cells in the presence of a known amount of IL-12 without said sample;
whereby the presence of neutralizing anti-IL12 antibodies in said wells is indicated by a reduction in the level of secreted IFN-γ in said wells as compared to said control IFN-γ level.

27. The method according to claim 28, wherein said sample dilutions are single point or serial dilutions.
